(19) United States
(12) Patent Application Publication
(10) Pub. No.: US 2020/0170881 A1
(43) Pub. Date: Jun. 4, 2020

(54) DEVICE FOR TREATING CARPAL TUNNEL SYNDROME

(71) Applicants: Yuval AVNI, Tel Aviv (IL); VINCENT YUVAL R&D LIMITED, Hong Kong (CN)

(72) Inventor: Yuval AVNI, Tel Aviv (IL)

(21) Appl. No.: 16/616,475

(22) PCT Filed: May 24, 2018
(86) PCT No.: PCT/IL2018/050563

§ 371 Date: Nov. 24, 2019

Related U.S. Application Data
(60) Provisional application No. 62/510,762, filed on May 25, 2017.

(51) Int. Cl. A61H 23/02; A61F 5/01; A61F 5/34

(52) U.S. Cl. CPC A61H 23/0263; A61F 5/0118; A61F 5/34; A61H 2205/06; A61H 2201/0103; A61H 2201/1676; A61H 2201/1638

(57) ABSTRACT

A device for treating a carpal tunnel syndrome comprises: (a) a brace configured for at least partially receiving a patient's hand; (b) displaceable support members mounted on the first member and spaced apart from each other and configured for supporting the patient's palm at areas adjacent to first and fifth metacarpal bones the patient's hand; and (c) a pressing member mounted on the second member and configured for adjustable pressure on dorsum of the patient's hand; The two displaceable support members and pressing member are mountable symmetrically to each other on palm and dorsum of the patient's hand to be received. The device comprises a generator of mechanical vibrations mounted between the displaceable support members. The vibrator is configured to apply the mechanical vibration in an area of carpal tunnel of the patient's hand.

### FIELD OF THE INVENTION

The present invention relates to orthopedic therapeutic devices, and, more specifically, to a device for treating the carpal tunnel syndrome.

### BACKGROUND OF THE INVENTION

Carpal tunnel syndrome is the result of a compromised or narrowed carpal canal leading to compression injury of the median nerve in the wrist. The carpal tunnel is the canal in the wrist through which the median nerve and flexor tendons pass from the forearm to the hand.

Carpal tunnel syndrome is caused by a deleterious increase in pressure on the median nerve which passes through the carpal tunnel (or canal) in the hand, adjacent 10 the wrist. The deleterious increase in pressure, which is brought on by prolonged repetitive motion of the hand and digits, is often caused by inflammation or damage to tendons for the hand which pass through the carpal tunnel along with the median nerve. Pressure increases can also be caused by narrowing of the carpal canal and by generalized swelling of the structures in the hand.

U.S. Pat. No. 5,702,355 discloses a portable appliance for treating carpal tunnel syndrome and/or other problems of the wrist of a person. The appliance comprises a releasably securable sleeve means for securement to a person's forearm, a stationary member positioned on the sleeve means, a moveable member slidably coupled to the stationary member, a cuff means secured to an end of the moveable member and a bias means coupled between the stationary member and the moveable member. The bias means is provided for urging the moveable member to slide from a retracted position to an extended position thus causing the cuff means to securely engage a portion of the person's hand adjacent the person's wrist to apply a predetermined tensile load to the person's wrist in a direction parallel to the longitudinal axis of the forearm.

U.S. Pat. No. 6,146,347 discloses a method and appliance for treating carpal tunnel syndrome having a splint with dorsal, thenar and hypothenar portions for applying pressure to the respective dorsal aspect of the hand and to thenar and hypothenar areas of a palmar aspect of the hand to induce stretching of the carpal ligament and flexor retinaculum. The thenar and hypothenar portions of the splint are connected to opposite sides of the dorsal portion and are spaced from the dorsal portion to allow for the insertion of the hand to be treated. The splint is preferably formed from a unitary piece of substantially rigid material and is sized and shaped to closely receive the hand with the dorsal, thenar and hypothenar portions substantially aligned with and covering the respective dorsal aspect, and thenar and hypothenar areas of the palmar aspect of the hand. An inflatable bladder, connected to a controllable fluid source, is located to contact, the dorsal aspect of the hand between the thenar and hypothenar areas of the palmar aspect to induce stretching of the carpal ligament and flexor retinaculum. A pressure gauge is connected to the bladder for monitoring. The controllable source of pressurized fluid is adjustable during treatment. The thenar portion of the splint can be releasably attachable with securing means to aid in insertion of the hand into the splint. In the method of use, the hand is inserted into the splint and the bladder is pressurized to apply pressure and induce stretching. The pressure is maintained at a constant or varying level for a predetermined period of time.

U.S. Pat. No. 6,953,440 discloses thee apparatus which stretches the carpal ligament and the flexor retinaculum, as well as the superficial structures and muscles of the hand, in a safe manner under precise control of the patient or a healthcare professional. A first embodiment of the inventive apparatus includes a housing for receiving the patient's hand with pressure elements in contact with the hypothenar, thenar and central longitudinal dorsal portions of the patient's hand. Active pressure sources are provided for the pressure elements and connected to an automatic control unit. The control unit causes the inventive apparatus to delivered therapeutic treatment to the hand by selectively activating the active pressure sources causing the thenar and hypothenar regions of the hand to be pulled apart and upward around the dorsally positioned pressure element in accordance with program instructions indicative of a treatment plan.

U.S. Pat. No. 6,942,631 discloses a therapeutic device for applying graded compression to the hand to remedy the symptoms of carpal tunnel syndrome. The device is a passive therapy device which receives the palm of the hand and applies graduated pressure to the top of the hand to relieve carpal tunnel region of the hand by spreading the area of the carpal tunnel There are also disclosed a centralizing means which allows for accurate placement of the means for apply pressure to the top of the hand and also a means for further spreading and fixing the spread in the carpal tunnel region.

### SUMMARY OF THE INVENTION

It is hence one object of the invention to disclose a device for treating a carpal tunnel syndrome. The aforesaid device comprises: (a) a brace configured for at least partially receiving a patient's hand; the brace formed by a first member and a second member; (b) displaceable support members mounted on the first member and spaced apart from each other and configured for supporting the patient's palm at areas adjacent to first and fifth metacarpal bones the patient's hand; (c) a pressing member mounted on the second member and configured for adjustable pressure on dorsum of the patient's hand. The two displaceable support members and pressing member are mountable on palm and dorsum of the patient's hand to be received.

It is a core purpose of the invention to provide the device comprising at least one generator of mechanical vibrations mounted between the displaceable support members. The vibrator is configured to apply the mechanical vibration in the palm, preferably but not necessarily in proximity to area of carpal tunnel of the patient's hand. In one embodiment of the present invention, the generator comprises a plurality of vibrators interfering therebetween.

Another object of the invention is to disclose at least one of the displaceable support members selected from the group consisting of a first mechanically displaceable support member and a first inflatable balloon.

A further object of the invention is to disclose the pressing member selected from the group consisting of a second mechanically displaceable support member and a second inflatable balloon.

A further object of the invention is to disclose the at least one of the displaceable support members pivoted to the first internal carrying surface.

A further object of the invention is to disclose the pressing member driven by a manually movable screw.

A further object of the invention is to disclose the first internal carrying surface and second internal carrying surface which are parallel in a closed position and inclined to each other in an open position such that the patient's hand is placeable into the brace between the two displaceable support members and pressing member.

A further object of the invention is to disclose the generator of mechanical vibrations pressable against the patient's hand by a third inflatable balloon.

A further object of the invention is to disclose the at least one of the first, second and third balloons connectable to a source of a compressed fluid.

A further object of the invention is to disclose the source of a compressed fluid embedded in the brace.

A further object of the invention is to disclose the fluid which is air.

A further object of the invention is to disclose the device comprising at least one valve configured for alternately feeding the fluid into at least one of the first, second and third balloons and releasing the fluid out them.

A further object of the invention is to disclose the device comprising a control unit for controlling the at least one valve and the generator of mechanical vibrations.

A further object of the invention is to disclose the device comprising a forth balloon configured for extending fingers of the patient's hand by pressing thereon.

A further object of the invention is to disclose the first and second members are hingedly connected to each other in a lockable manner.

A further object of the invention is to disclose the displaceable support members displaceable by a mechanism comprising an inflatable member spacing apart the displaceable support members and a tension spring repositing thereof.

A further object of the invention is to disclose a device for treating a carpal tunnel syndrome. The aforesaid device comprises: (a) a brace configured for at least partially receiving a patient's hand; said brace formed by a first member and a second member interconnected therebetween; (b) at least two displaceable support first inflatable balloons mounted on said first member and spaced apart from each other and configured for supporting the patient's palm at areas adjacent to first and fifth metacarpal bones the patient's hand; (c) a second inflatable balloon mounted on said second member and configured for adjustable pressure on dorsum of the patient's hand. The two displaceable support members and pressing member are mountable on palm and dorsum of the patient's hand to be received.

A further object of the invention is to disclose a device for treating a carpal tunnel syndrome. The said device comprises (a) a brace configured for at least partially receiving a patient's hand; said brace formed by a first member and a second member interconnected therebetween; (b) displaceable support members mounted on said first member and spaced apart from each other and configured for supporting the patient's palm at areas adjacent to first and fifth metacarpal bones the patient's hand; (c) a pressing member mounted on said second member and configured for adjustable pressure on dorsum of the patient's hand. The two displaceable support members and pressing member are mountable on palm and dorsum of the patient's hand to be received; The first member comprises an actuator comprising a pneumatically operated inflatable member spacing apart said displaceable support members and a tension spring repositing thereof.

A further object of the invention is to disclose a method of treating a carpal tunnel syndrome. The aforesaid method comprises steps of: (a) providing a device further comprising: (i) a brace configured for at least partially receiving a patient's hand; the brace having a first internal carrying surface and a second internal carrying surface; (ii) displaceable support members mounted on the first internal carrying surface and spaced apart from each other and configured for supporting the patient's palm at areas adjacent to first and fifth metacarpal bones the patient's hand; (iii) a pressing member mounted on the second internal carrying surface and configured for adjustable pressure on dorsum of the patient's hand; the two displaceable support members and pressing member are mountable symmetrically to each other on palm and dorsum of the patient's hand to be received; the device comprises a generator of mechanical vibrations mounted between the displaceable support members; the vibrator is configured to apply the mechanical vibration in an area of carpal tunnel of the patient's hand; (b) placing the patient's middle carpal joint within the brace between the displaceable support members and pressing member; (c) applying pressure to palm and dorsum of the patient's hand by the two displaceable support members and pressing member; (d) applying vibrations to the area of carpal tunnel.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a side view of a device for treating a carpal tunnel syndrome;

FIGS. 2a and 2b are side views of a device for treating a carpal tunnel syndrome with stretching tendons by alternating position of the fingers;

FIG. 3 is a schematic side view of a mechanism for positioning pivoted support members;

FIGS. 4a and 4b are cross-sectional views of a device for treating a carpal tunnel syndrome;

FIGS. 5a and 5b cross-sectional views of two alternative embodiments of a device for treating a carpal tunnel syndrome; and

FIGS. 6a and 6b are cross-sectional views of an embodiment of a device for treating a carpal tunnel syndrome with a pneumatic mechanism for positioning support members.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a device for treating a carpal tunnel syndrome and a method of implementing the same.

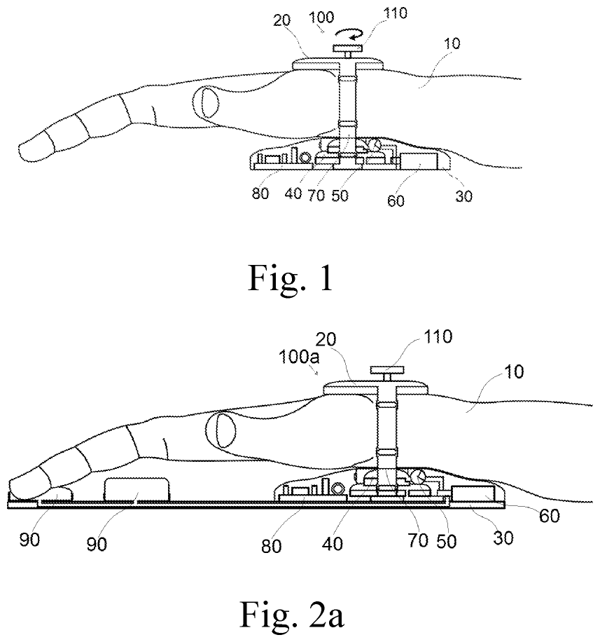

Reference is now made to FIG. 1 presenting device 100 for treating a carpal tunnel syndrome. Patient's hand 10 is placed between first member 30 and second member 20. Members 20 and 30 are movable relative to each other for needs of wearing, removing and individually fitting device 100 on the patient's hand. According one embodiment of the present invention, first member 30 carries pump 60, pneumatic supporting member 40 connected to pump 60, vibrator 50 and control unit 80. Second member 20 is provided with screw 110 displacing a pressing member (not shown) relative to second member 20. Control unit 80 is preprogrammed for applying pressure to the carpal tunnel area according a predetermined protocol defining a pressure value and time schedule of the treatment.

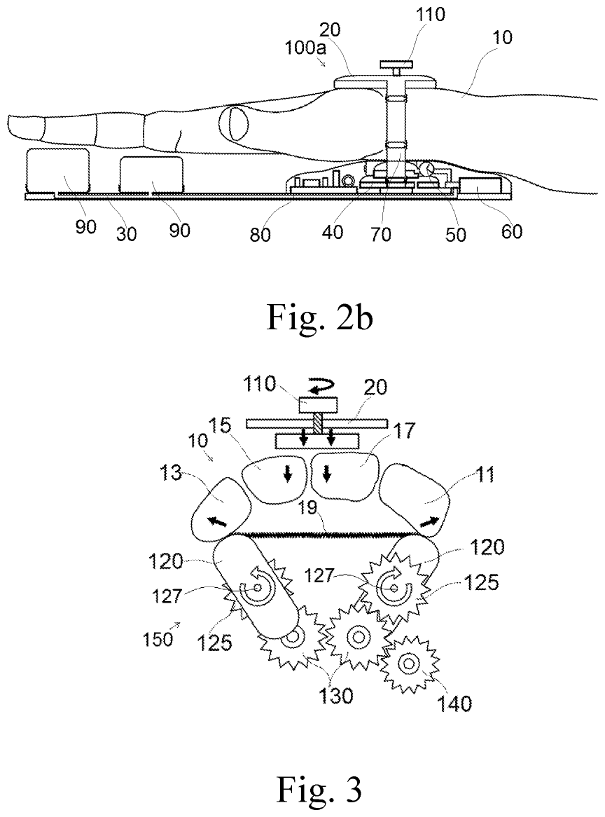

Reference is now made to FIGS. 2a and 2b showing alternative embodiment 100a of the present invention. Specifically, device 100a is provided with inflatable supporting members 90 designed for alternate stretching and releasing fingers of patient's hand 10. While FIG. 2a illustrates finger stretching when members 90 are inflated, FIG. 2b corresponds to a timeout phase with deflated members 90.

Reference is now made to FIG. 3 showing mechanism 150 of positioning pivoted support members 120. Specifically, patient's hand 10 is placed within the device for treating a carpal tunnel syndrome such that support members 120 rotatable around axes 127 contact with patient's hand 10 in locations of first and fifth metacarpal bones 11 and 13, respectively. Rotational torque is transferred from drive gear 140 to driven gear 125 via idlers 130. Screw 110 is designed for axial displacement of pressing member 115. The effect of mechanism 150 is designed for stretching transversal carpal ligament 19.

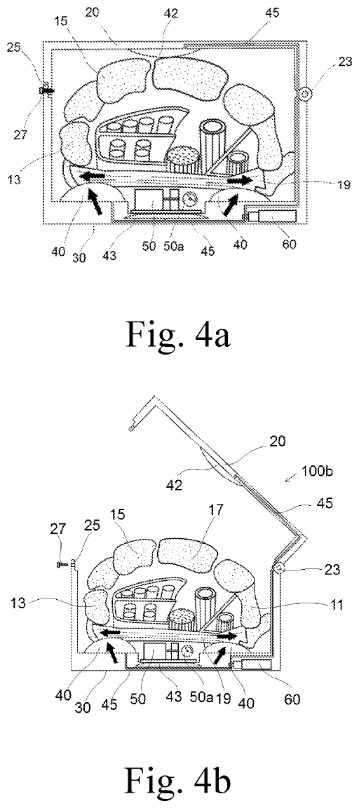

Reference is now made to FIGS. 4a and 4b showing pneumatic embodiment 100b of the present invention in open and closed positions. Supporting members 40 are embodied by first inflatable balloons 41. Numeral 41 refers to second inflatable balloon functioning as pressing member 115 (FIG. 3). Third balloon 43 is configured for keeping snug vibrator 50 against the carpal tunnel area of patient's hand 10. According to one embodiment of the present invention, device 100b comprises vibrator 50a interfering with vibrator 50. Vibrators 50 and 50a can be embodied as eccentric vibrators driven by electric motors.

Pump 60 controlled by control unit 80 (not shown) inflates first, second and third balloons 41, 42 and 43, respectively, according to a predetermined protocol. Concurrent and alternate inflation and deflation is in the scope of the present invention. Device 100b provides treatment of mechanical pressure and vibration in a combined manner. Upper and lower members 20 and 30 are connected to each other by hinge 23. Members 20 and 30 can be locked by a rod 27 insertable into ear 25. Effect of stretching transversal carpal ligament 15 is increased by applying vibration.

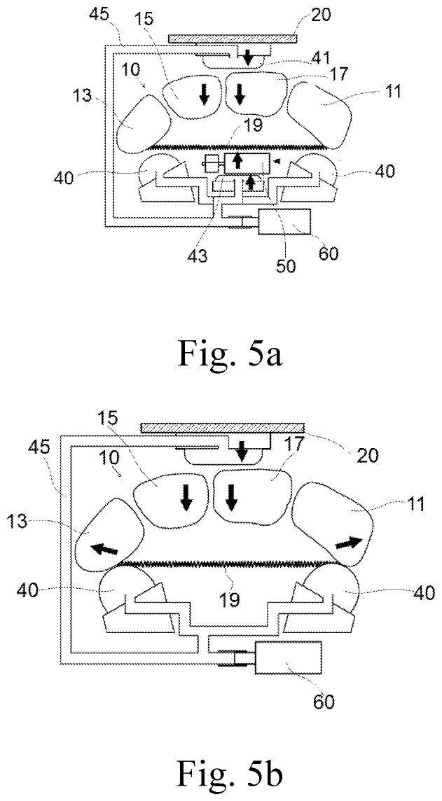

Reference is now made to FIGS. 5a and 5b illustrating the principle of operation of two embodiments of the present invention. The embodiment presented in FIG. 5a is provided with vibrator 50.

Numerals 11, 13, 15 and 17 refer to schematically shown bones of patient's hand 10. The device of the present invention is designed for stretching tendon 19. An eccentric vibrator and a combination of interfering eccentric vibrators are in the scope of the present invention. While bones 11 and 13 are supported by balloons 40, balloon 41 applies pressure to bones 15 and 17 from above. As a result, tendon 19 is stretched.

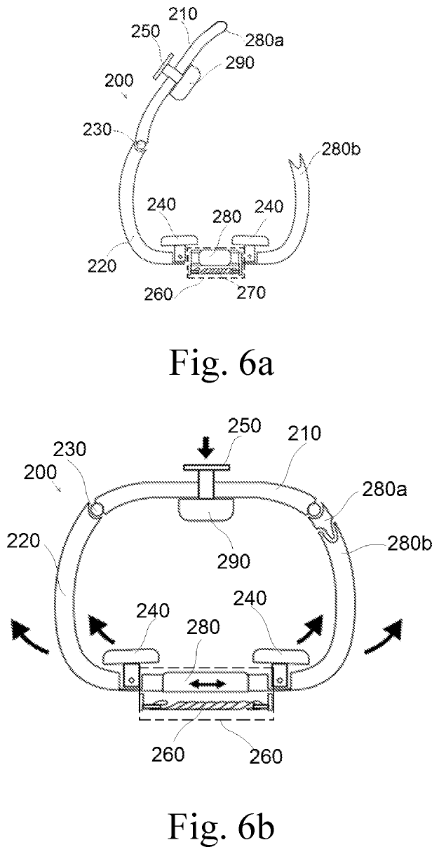

Reference is now made to FIGS. 6a and 6b presenting an alternative embodiment 200. Device 200 comprises first and second members 210 and 220 connected to each other by hinge 230. Terminals 280a and 280b are lockable to each other. Supporting members 240 are displaceable by mechanism 260 comprising an inflatable member 270 spacing apart support members 240 and a tension spring which reposits thereof support members 240. Pressing member 290 is displaceable by screw 250.

1.-25. (canceled)

26. A device for treating a carpal tunnel syndrome; said device comprising:
a. a brace configured for at least partially receiving a patient's hand; said brace formed by a first member and a second member interconnected therebetween;
b. displaceable support members mounted on said first member and spaced apart from each other and configured for supporting the patient's palm at areas adjacent to first and fifth metacarpal bones the patient's hand;
c. a pressing member mounted on said second member and configured for adjustable pressure on dorsum of the patient's hand;
said two displaceable support members and pressing member are mountable on palm and dorsum of the patient's hand to be received;
wherein said device comprises at least one eccentric vibrator mounted between said displaceable support members; said at least one eccentric vibrator is configured to apply said mechanical vibration in a palm's area of carpal tunnel of the patient's hand.

27. The device according to claim 26, wherein at least one of said displaceable support members is selected from the group consisting of a first mechanically displaceable support member and a first inflatable balloon.

28. The device according to claim 27, wherein said first is connectable to a source of a compressed fluid.

29. The device according to claim 28, wherein said source of a compressed fluid is embedded in said brace.

30. The device according to claim 26, wherein said pressing member is selected from the group consisting of a second mechanically displaceable support member and a second inflatable balloon.

31. The device according to claim 30, wherein said second balloon is connectable to a source of a compressed fluid.

32. The device according to claim 31, wherein said source of a compressed fluid is embedded in said brace.

33. The device according to claim 37, wherein said at least one of said displaceable support members is pivoted to said first internal carrying surface.

34. The device according to claim 30, wherein said pressing member is driven by a manually movable screw.

35. The device according to claim 26, wherein said first internal carrying surface and second internal carrying surface are parallel in a closed position and inclined to each other in an open position such that the patient's hand is placeable into said brace between said two displaceable support members and pressing member.

36. The device according to claim 26, wherein said first and second members are releasably connectable.

37. The device according to claim 26, wherein at least one eccentric vibrator is pressable against the patient's hand by a third inflatable balloon.

38. The device according to claim 37, wherein said first member is connectable to a source of a compressed fluid.

39. The device according to claim 38, wherein said source of a compressed fluid is embedded in said brace.

40. The device according to claim 37, wherein at least one of the following is true:
a. said device comprises at least one valve configured for alternately feeding said fluid into at least one of said first, second and third balloons and releasing said fluid out of at least one of said first, second and third balloons;
b. said device comprises a control unit for controlling said at least one valve and said at least one eccentric vibrator; and
c. said device comprises a forth balloon configured for extending fingers of the patient's hand by pressing thereon.

41. The device according to claim 26, wherein said first and second members are hingedly connected to each other in a lockable manner.

42. The device according to claim 26, wherein said displaceable support members are displaceable by a mechanism comprising an inflatable member spacing apart said displaceable support members and a tension spring repositing thereof.

43. A method of treating a carpal tunnel syndrome; said method comprising steps of:
a. providing a device further comprising:
i. a brace configured for at least partially receiving a patient's hand; said brace having a first internal carrying surface and a second internal carrying surface;
ii. displaceable support members mounted on said first internal carrying surface and spaced apart from each other and configured for supporting the patient's palm at areas adjacent to first and fifth metacarpal bones the patient's hand;
iii. a pressing member mounted on said second internal carrying surface and configured for adjustable pressure on dorsum of the patient's hand;
said two displaceable support members and pressing member are mountable symmetrically to each other on palm and dorsum of the patient's hand to be received;
said device comprises at least one eccentric vibrator configured to apply said mechanical vibration in an area of carpal tunnel of the patient's hand;
b. placing the patient's middle carpal joint within said brace between said displaceable support members and pressing member;
c. applying pressure to palm and dorsum of the patient's hand by said two displaceable support members and pressing member.
d. applying vibrations to the area of carpal tunnel.
